(19) United States
(12) Patent Application Publication
(10) Pub. No.: US 2013/0338433 A1
(43) Pub. Date: Dec. 19, 2013

(54) RAPIDLY DEPLOYABLE FLEXIBLE ROBOTIC INSTRUMENTATION

(71) Applicants: Roger Goldman, New York, NY (US); Nabil Simaan, Nashville, TN (US); Mitchell C. Benson, New York, NY (US); Lara S. MacLachlan, New York, NY (US)

(72) Inventors: Roger Goldman, New York, NY (US); Nabil Simaan, Nashville, TN (US); Mitchell C. Benson, New York, NY (US); Lara S. MacLachlan, New York, NY (US)

(21) Appl. No.: 13/812,441

(22) PCT Filed: Jul. 26, 2011
(86) PCT No.: PCT/US11/45357

§ 371 Date: Sep. 3, 2013

Related U.S. Application Data
(60) Provisional application No. 61/368,193, filed on Jul. 27, 2010.
(60) Provisional application No. 61/470,730, filed on Apr. 1, 2011.

(51) Int. Cl. A61B 19/00; A61B 1/00

(52) U.S. Cl. CPC A61B 19/2203; A61B 1/00149
USPC 600/102; 600/104

(57) ABSTRACT

A robotic system and method are provided. The robotic system includes a continuum robot, an actuation unit, and a flexible positioning shaft. The continuum robot is configured to perform minimally invasive diagnostic, surgical or therapeutic techniques, and includes at least one continuum segment including a plurality of backbones. The continuum segment carries at least one diagnostic, surgical or therapeutic instrument in a flexible instrumentation housing that has a plurality of instrumentation channels. The actuation unit is configured to actuate the continuum robot by providing linear actuation to each of the plurality of backbones, and includes force sensors for measuring actuation forces. The flexible positioning shaft is configured to direct a position and orientation of the continuum robot and to couple the actuation unit to the continuum robot.

### CROSS-REFERENCE TO RELATED APPLICATIONS

This application claims the benefit under 35 U.S.C. §119(e) of U.S. Provisional Patent Application No. 61/368,193, filed on Jul. 27, 2010 and U.S. Provisional Patent Application No. 61/470,730, filed on Apr. 1, 2011, each of which is hereby incorporated by reference herein in its entirety.

### BACKGROUND

In conventional surgery, a surgeon has to cut openings in a patient large enough to allow visualization of and manual access to the surgical site. In the past two decades, however, medical surgery has steadily advanced to include Minimally Invasive Surgery (MIS), which includes surgical techniques that are less invasive than conventional surgery.

Minimally invasive diagnostic and/or surgical procedures benefit patients with reduced trauma and faster healing time by providing doctors and surgeons with access to internal organs via a limited number of small incisions in a patient's body. Typically, cannulas or sleeves are inserted through small incisions to provide entry ports through which surgical instruments are passed. These access ports, however, constrain instruments for the procedures to only four degrees-of-freedom (DoFs) and limit their distal dexterity. To help doctors and surgeons overcome these difficulties, a large number of robotic devices and systems have been designed for many minimally invasive procedures.

Despite the large number of previous works, however, current robotic instruments are still too large and/or have insufficient dexterity for some clinical applications. For instance, clinical applications characterized by deep and narrow diagnostic/surgical fields, such as neurosurgery, fetal surgery, and transurethral resection of bladder tumors, are beyond the capabilities of existing commercial diagnostic/surgical systems owing to size and dexterity limitations.

In addition, current robotic instruments require a long pre-operative and intra-operative preparation for deployment. Deploying the robotic instruments for a surgery, for instance, requires the instruments to be precisely positioned in the operating room before the patient is brought in and then still further adjusted to orient the instruments towards the surgical site after the patient is brought in to the operating room. This pre-operative preparation can be cumbersome for pre-scheduled surgeries, and it is a critical bottleneck for the surgeries that cannot be pre-scheduled, such as emergency operations. In fact, surgeons in emergency operations are often left with no choice but to revert to conventional open surgery due to unacceptable delays in deploying the robotic instruments.

### SUMMARY

Rapidly deployable flexible robotic systems and methods are provided. The disclosed subject matter allows for rapid deployment of flexible robotic instrumentation for minimally invasive diagnosis and intervention. The disclosed subject matter also facilitates minimally invasive surgery in deep surgical sites where rigid manual instruments, such as rigid endoscopy and laparascopy equipment, are cumbersome, or unable to navigate around and gain access to targeted tissues or organs.

In one embodiment, a robotic system is provided. The robotic system includes a continuum robot, an actuation unit, and a flexible positioning shaft. The continuum robot is configured to perform minimally invasive diagnostic, surgical or therapeutic techniques, and includes at least one continuum segment including a plurality of backbones. The continuum segment carries at least one diagnostic, surgical or therapeutic instrument in a flexible instrumentation housing that has a plurality of instrumentation channels. The actuation unit is configured to actuate the continuum robot by providing linear actuation to each of the plurality of backbones, and includes force sensors for measuring actuation forces. The flexible positioning shaft is configured to direct a position and orientation of the continuum robot and to couple the actuation unit to the continuum robot.

In another embodiment, a method for deploying a robotic device is provided. The method includes: providing a robotic device including an actuation unit, a flexible positioning shaft, and a continuum robot that is actuated by the actuation unit for performing minimally invasive procedures, wherein the robotic device is mounted on a linear stage and wherein the flexible positioning shaft is configured to couple the actuation unit to the continuum robot; positioning the robotic device with respect to a surgical bed; adjusting the flexible positioning shaft to orient the robotic device towards an entry to a targeted surgical site; and inserting the robotic device into the entry by advancing the linear stage.

In yet another embodiment, a robotic system for minimally invasive urologic procedures is provided. The robotic system includes a continuum robot, an actuation unit, and a flexible shaft section. The continuum robot is configured for performing minimally invasive urologic procedures and includes a proximal continuum segment that is serially coupled to a distal continuum segment. The serially coupled segments include a plurality of backbones and carry at least one diagnostic, surgical or therapeutic instrument. The actuation unit is configured for actuating the continuum robot by providing linear actuation to each of the plurality of backbones and includes force sensors for measuring actuation forces. The flexible shaft section is configured for directing a position and orientation of the continuum robot and for coupling the actuation unit to a transurethral resectoscope. The transurethral resectoscope guides a flexible instrument housing and the at least one instrument from the actuation unit to the continuum robot. An adjustment arm rigidly anchors a proximal end and a distal end of the flexible shaft for adjusting the flexible shaft section to a desired position and orientation.

### BRIEF DESCRIPTION OF THE DRAWINGS

FIG. 1 is an illustration of a rapidly deployable flexible robotic system for minimally invasive diagnosis and surgery in accordance with some embodiments of the disclosed subject matter.

FIG. 2 is an illustration of a flexible instrumentation housing that may be deployed by a rapidly deployable flexible robotic system in accordance with some embodiments of the disclosed subject matter.

FIGS. 3A-B are illustrations of a rapidly deployable flexible robotic system for minimally invasive diagnosis and surgery in accordance with some embodiments of the disclosed subject matter.

FIG. 4 is an illustration showing individual components and subassemblies of an actuation unit included in a rapidly deployable flexible robotic system in accordance with some embodiments of the disclosed subject matter.

FIG. 5 is an illustration showing a cross section view of a concentric backbone actuation assembly in accordance with some embodiments of the disclosed subject matter.

FIG. 6A is an illustration of a continuum robot having two serially stacked continuum segments in accordance with some embodiments of the disclosed subject matter.

FIG. 6B is an illustration showing a cross section view of a continuum segment in accordance with some embodiments of the disclosed subject matter.

FIG. 7 is an illustration showing a structure and kinematics nomenclature for a continuum segment in accordance with some embodiments of the disclosed subject matter.

FIG. 8A is an illustration of a rapidly deployable flexible robotic system for minimally invasive diagnosis and surgery in accordance with some embodiments of the disclosed subject matter.

FIG. 8B is an illustration showing a cross section view of an adjustment shaft of a rapidly deployable flexible robotic system in accordance with some embodiments of the disclosed subject matter.

FIG. 9A is an illustration of a rapidly deployable flexible robotic system for minimally invasive urologic procedures in accordance with some embodiments of the disclosed subject matter.

FIG. 9B is an illustration showing a cross section view of a flexible positioning shaft of a rapidly deployable flexible robotic system for minimally invasive urologic procedures in accordance with some embodiments of the disclosed subject matter.

FIG. 10 is an illustration showing a deployment of a rapidly deployable flexible robotic system for performing transurethral resection of bladder tumors (TURBT) in accordance with some embodiments of the disclosed subject matter.

FIG. 11 is an illustration showing a cross section view of the workspace that is reachable by a continuum robot of a rapidly deployable flexible robotic system for minimally invasive urologic procedures in accordance with some embodiments of the disclosed subject matter.

FIG. 12 is a flow chart for a method for deploying a robotic device for minimally invasive diagnosis and surgery in accordance with some embodiments of the disclosed subject matter.

### DETAILED DESCRIPTION

Rapidly deployable flexible robotic systems and methods are provided. In some embodiments of the disclosed subject matter, rapidly deployable flexible robotic systems are provided for minimally invasive diagnosis and surgery in deep surgical sites where rigid manual instrumentation is unable to navigate around or gain access to targeted tissues or organs.

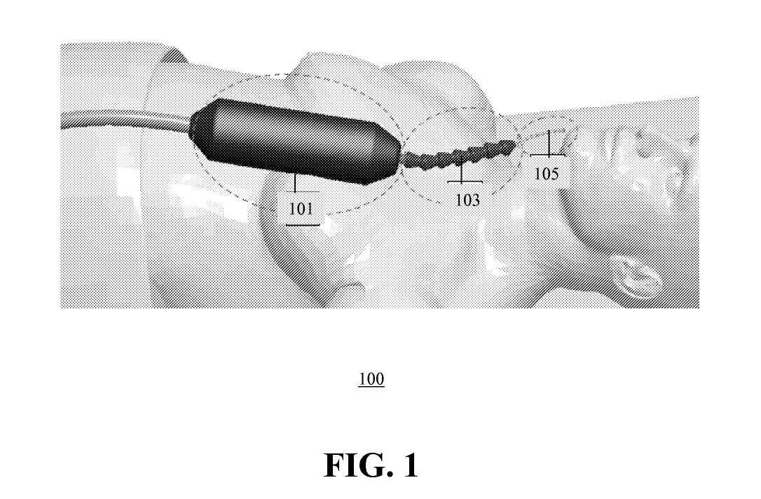

FIG. 1 is an illustration of a rapidly deployable flexible robotic system 100 for minimally invasive diagnosis and surgery in accordance with some embodiments of the disclosed subject matter. Referring to FIG. 1, rapidly deployable flexible robotic system 100 includes an actuation unit 101, a flexible and lockable, manually-adjustable, positioning shaft 103, and an insertable continuum robot 105. In some embodiments, actuation unit 101 may incorporate seven degrees of freedom (DoF) with the capability to control two serially coupled (e.g., proximal segment, distal segment) redundant three-backbone continuum segments with an advancement-retraction stage for semi-automated insertion. In some embodiments, force sensors on each independent backbone of the continuum segments may measure the backbone actuation forces. The sensed actuation forces may also allow estimation of the distal environment interaction forces and compliant motion algorithms.

In some embodiments, adjustable positioning shaft 103 may allow rapid positioning of continuum robot 105 in a manner in which surgical and/or diagnostic instruments can be brought in and out of the surgical workflow rapidly. In some embodiments, adjustable positioning shaft 103 may use an internal cable tension that is controlled either manually or by computer such that the cable can be loosened when manual repositioning of adjustable positioning shaft 103 is desired and tightened when repositioning is complete to lock in the newly positioned configuration.

In some embodiments, bending section of continuum robot 105 can be extended or retracted from the distal end of adjustable positioning shaft 103. In some embodiments, continuum robot 105 may be deployed via a master interface coupled to a master console that includes a display and an interface for controlling continuum robot 105 and inserted instrumentation.

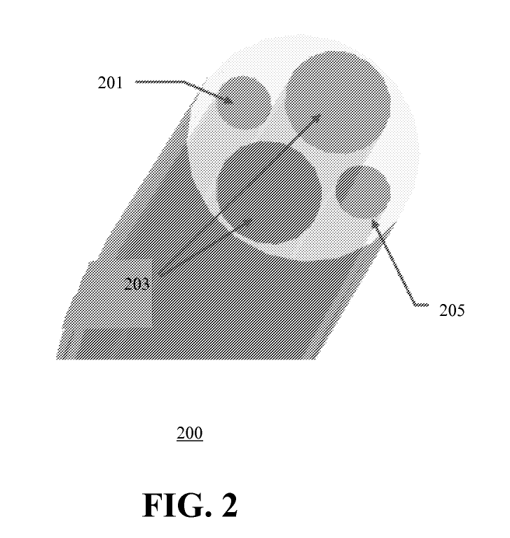

FIG. 2 is an illustration of a flexible instrumentation housing 200 that may be deployed by continuum robot 105 in accordance with some embodiments of the disclosed subject matter. Referring to FIG. 2, flexible instrumentation housing 200 includes a plurality of independent lumens 201, 203, and 205. In some embodiments, flexible instrumentation housing 200 includes at least one lumen, such as lumens 201 and 205, for carrying lighting and/or imaging instruments for, e.g., white-light based endoscopy. In some embodiments, lumens 201 and 205 are measured 0.55 mm in diameter. In some embodiments, flexible instrumentation housing 200 includes at least one lumen, such as lumens 203, for carrying microsurgical instruments, such as cold biopsy forceps and resection loop. In some embodiments, lumens 203 are measured 1.2 mm in diameter. In some embodiments, independent lumens 201, 203, and 205 are supported by a lubricious, poly-tetrafluoroethylene (PTFE) structure.

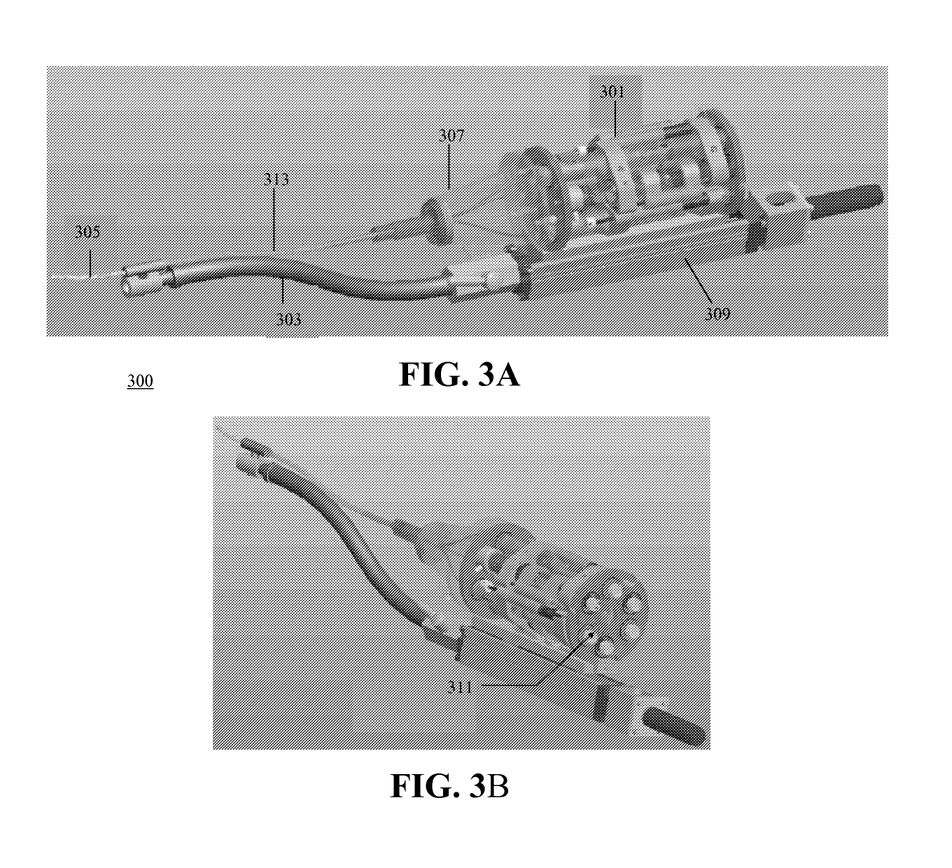

FIGS. 3A-B are illustrations of a rapidly deployable flexible robotic system 300 for minimally invasive diagnosis and surgery in accordance with some embodiments of the disclosed subject matter. Referring to FIGS. 3A-B, rapidly deployable flexible robotic system 300 includes an actuation unit 301, a lockable, manually-adjustable, positioning shaft 303, a three-backbone continuum robot 305 having two serially coupled continuum segments with an advanced-retraction stage, a cone assembly 307, a linear insertion stage 309, a flexible instrumentation housing 313, and a proximal end of an instrumentation channel 311. In some embodiments, actuation unit 301 is mounted to linear insertion stage 309.

In some embodiments, actuation unit 301 may be a six DoF bundled actuation unit with integrated force sensors. In some embodiments, actuation unit 301 includes three concentric backbone actuation assemblies, each of which includes coupled actuation cylinders. Each of the cylinders actuates one of the six total actuation lines in continuum robot 305. In some embodiments, for instance, each coupled stage includes a primary and a secondary cylinder and actuates the proximal and distal segments of each backbone in continuum robot 305.

In some embodiments, cone assembly 307 routes the actuation lines of continuum robot 305 from actuation unit 301 into flexible instrumentation housing 313 that guides the actuation lines and instrumentation to continuum robot 305. In some embodiments, the instrumentation is inserted into the flexible housing through the proximal end of instrumentation channel 311.

In some embodiments, adjustable positioning shaft 303 provides a manually adjustable flexible section that directs the position and orientation of continuum robot 305. Adjustable positioning shaft 303 enables rapid deployment of robotic system 300 in the operating room by providing the capability to change the position and orientation of robotic system 300, thereby reducing the amount of time required to prepare the system for, e.g., a surgery.

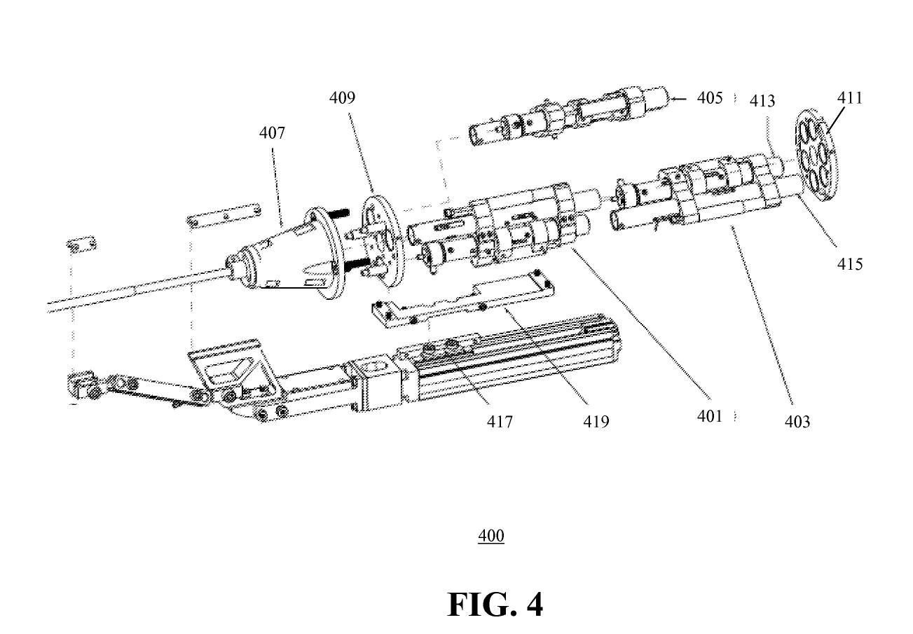

FIG. 4 is an illustration showing an exploded view of an actuation unit 400 included in a rapidly deployable flexible robotic system in accordance with some embodiments of the disclosed subject matter. Referring to FIG. 4, actuation unit 400 includes concentric backbone actuation assemblies 401, 403, and 405, a cone assembly 407, and a set of base and top plates 409 and 411. Each backbone actuation assembly includes a primary cylinder 413 and a secondary cylinder 415, which connects to base and top plates 409 and 411.

In some embodiments, actuation unit 400 is mounted on an insertion stage 417 through a connection plate 419. In some embodiments, the backbones of a continuum robot coupled to actuation unit 400 are routed from concentric actuation assemblies 401, 403, and 405 through the backbone spacing cone assembly 407 to the continuum robot.

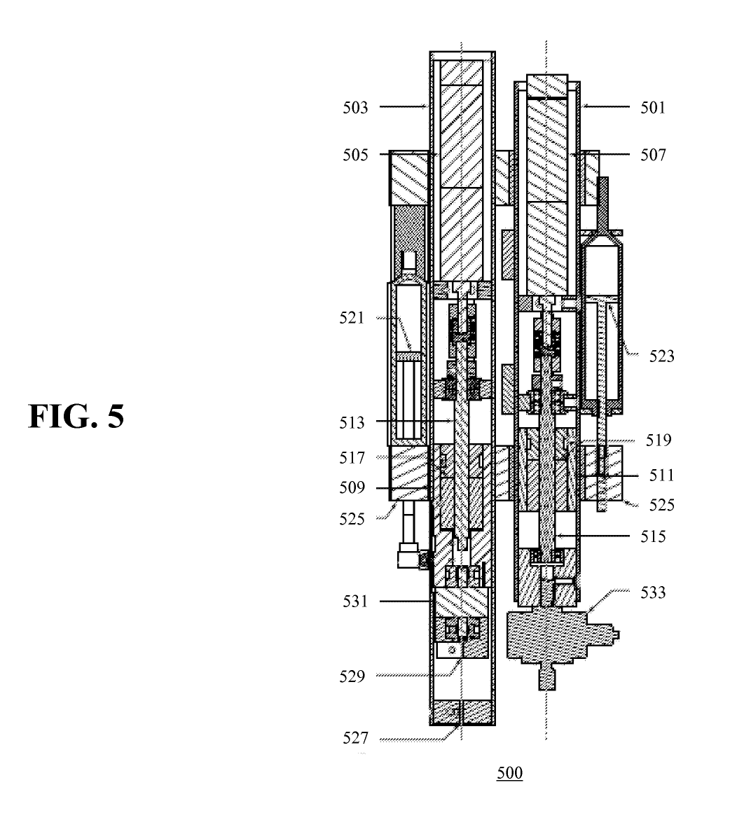

FIG. 5 is an illustration showing a cross section view of a concentric backbone actuation assembly 500 in accordance with some embodiments of the disclosed subject matter. Referring to FIG. 5, concentric backbone actuation assembly 500 includes a primary cylinder 501 and a secondary cylinder 503. Each cylinder, both primary and secondary, contains a motor 505 or 507 that drives a piston 509 or 511 by an internal lead screw 513 or 515.

In some embodiments, each of lead screw nuts 517 and 519 inside respective pistons 509 and 511 includes two elements that can be tightened on lead screw 513 or 515 with respect to each other to remove backlash between piston 509 or 511 and lead screw 513 or 515. In some embodiments, linear motion of pistons 509 and 511 can be dually measured by motor encoders, which are integrated into motors 505 and 507, and linear potentiometers 521 and 523. In some embodiments, primary cylinder piston 511 is rigidly connected by shear pins to a connection arm 525 that is clamped to the outside diameter of secondary cylinder 503.

In some embodiments, the secondary backbones of the proximal segment of a continuum robot is connected to a base 527 of secondary cylinder 503, thereby the motion of primary cylinder piston 511 drives the proximal secondary backbone relative to primary cylinder 501. In some embodiments, secondary cylinder piston 509 is attached to the connection of a secondary backbone wire 529 of the distal segment of a continuum robot through a secondary cylinder load cell 531. Secondary cylinder load cell 531 can directly measure actuation forces in the distal secondary backbones of the continuum robot.

In some embodiments, concentric backbone actuation assembly 500 is connected to the base plate of an actuation unit through a primary cylinder load cell 533. Primary cylinder load cell 533 can measure the sum of the actuation forces in a set of coaxial secondary backbones attached to the assembly. In some embodiments, concentric backbone actuation assembly 500 is supported on nylon bushings at the proximal end of primary and secondary cylinders 501 and 503 to prevent moments on primary cylinder load cell 533.

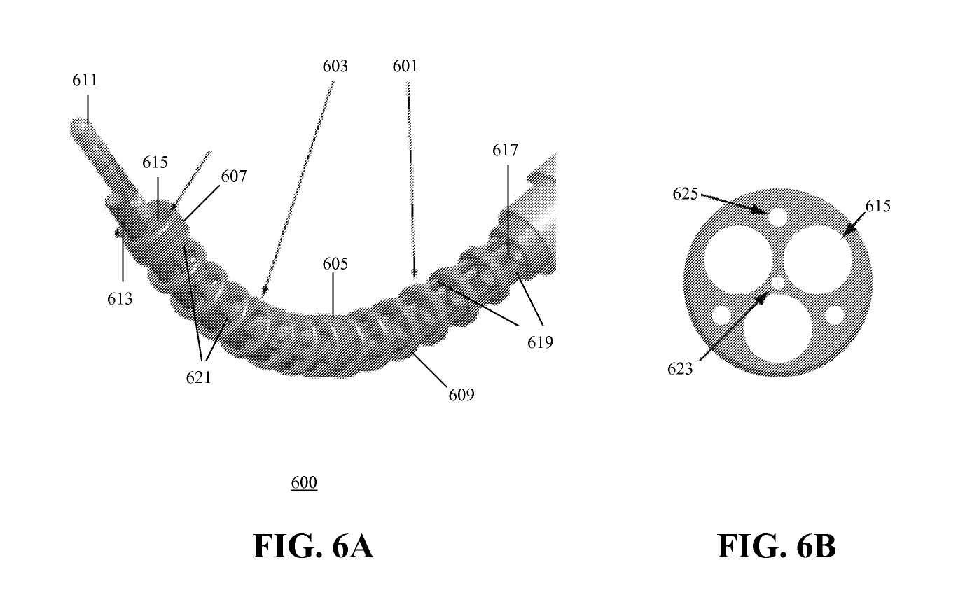

FIG. 6A is an illustration of a continuum robot 600 having two serially stacked continuum segments in accordance with some embodiments of the disclosed subject matter. Referring to FIG. 6A, continuum robot 600 includes a proximal continuum segment 601 and a distal continuum segment 603.

Each of segments 601 and 603 is constructed from one centrally located passive primary backbone 617 and three radially symmetric, actuated secondary backbones 619 and 621 that are bounded by end disks 605 (proximal segment end disk) or 607 (distal segment end disk) and a multitude of spacer disks 609, which maintain approximate radial symmetry as the segment moves through a workspace. FIG. 6B shows a cross section view of continuum segments 601 and 603 in accordance with some embodiments of the disclosed subject matter.

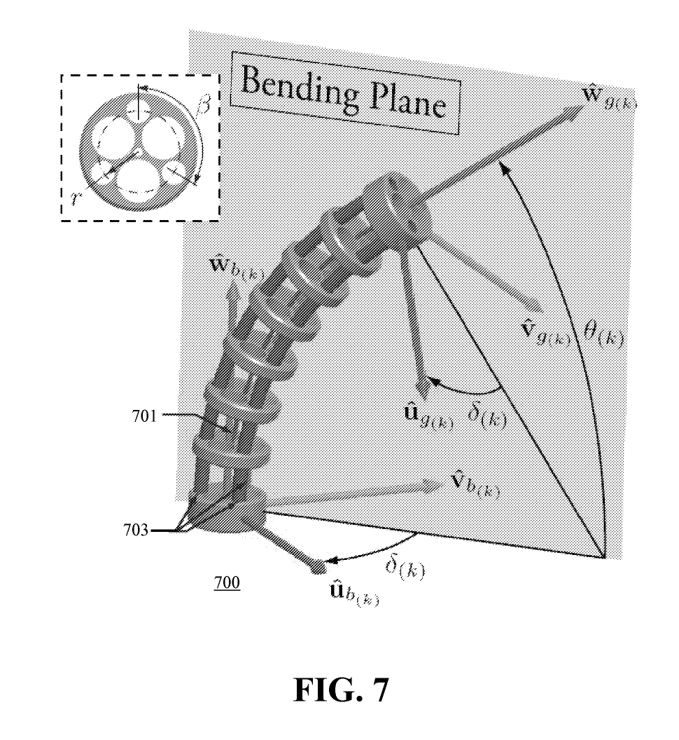

In some embodiments, secondary backbones 619 (proximal secondary backbones) and 621 (distal secondary backbones) are equally spaced with a separation angle β (shown in FIG. 7) and a pitch circle radius r from primary backbone 617. In some embodiments, proximal secondary backbones 619 are superelastic nitinol tubes. In some embodiments, distal secondary backbones 621 are nitinol wires and run inside proximal secondary backbones 619. In some embodiments, primary backbone 617 is nitinol wire.

Continuum robot 600 provides a set of instrumentation channels 615 for carrying surgical instruments, such as a biopsy forceps 611, and visualization instruments, such as a fiberscope 613. In some embodiments, fiberscope 613 is a flexible 1 mm diameter fiberscope with a 10 k pixel fused image guide. In some embodiments, fiberscope 613 is coupled to a camera system.

Referring to FIG. 6B, a cross section of continuum segments 601 and 603 contains three equally spaced instrument channels (also referred to as instrument lumens) 615, a primary backbone lumen 623, and secondary backbone lumens 625.

FIG. 7 is an illustration showing a structure and kinematics nomenclature for a continuum segment 700 in accordance with some embodiments of the disclosed subject matter. Referring to FIG. 7, the pose of a k^{th} segment multi-backbone continuum robot can be described in a set of generalized coordinates by a configuration space vector defined as

ψ₍ₖ₎=[θ₍ₖ₎,δ₍ₖ₎]^{T} (1)

where (•)₍ₖ₎ for k=1, 2, . . . denotes a variable associated with the k^{th} segment and θ₍ₖ₎ and δ₍ₖ₎ define respectively the bending angle and the orientation of the bending plane of the segment.

The inverse kinematics relating the configuration space, ψ₍ₖ₎, to the joint space

q₍ₖ₎=[q_{1,(k)}, . . . ,q_{m,(k)}]^{T}

is given by

L_{j,(k)}=L₍ₖ₎+q_{j,(k)}=L₍ₖ₎+Δ_{j,(k)}Θ₍ₖ₎. j=1, . . . ,m (2)

where L_{j,(k)} is the length of the j^{th} secondary backbone 703 of the k^{th} segment, L₍ₖ₎ is the length of the primary backbone 701 of the k^{th} segment,

$${Δ}_{j , ( k )} = r cos {σ}_{j , ( k )} , {σ}_{j , ( k )} = {δ}_{( k )} + ( j - 1 ) \frac{2 π}{3} , and {Θ}_{( k )} = {θ}_{( k )} - \frac{π}{2} .$$

The instantaneous inverse kinematics can be described by differentiating equation (2) to yield

\dot{q}₍ₖ₎=J_{qψ}_{₍ₖ₎}\dot{ψ}₍ₖ₎ (3)

where the Jacobian J_{qψ}_{₍ₖ₎} is given by

$${J}_{q {ψ}_{( k )}} = [ \begin{matrix}r {c}_{{σ}_{1 , ( k )}} & - r {Θ}_{( k )} {s}_{{σ}_{1 , ( k )}} \\ ⋮ & ⋮ \\ r {c}_{{σ}_{m , ( k )}} & - r {Θ}_{( k )} {s}_{{σ}_{m , ( k )}}\end{matrix} ] \qquad ( 4 )$$

where c_{α} [symbol] cos(α) and s_{α} [symbol] sin(α).

The direct kinematics of the k^{th} segment is given by the position ^{b}^{₍ₖ₎}P_{b}_{₍ₖ₎}_{g}_{₍ₖ₎} and orientation ^{b}^{₍ₖ₎}R_{g}_{₍ₖ₎} of the segment end disk with respect to its base disk. For

$${θ}_{( k )} \neq \frac{π}{2} ,$$

the kinematics takes the form

$$\begin{matrix}{}_{}^{{b}_{( k )}}{P}{}_{{b}_{( k )} {g}_{( k )}}^{} = \frac{{L}_{( k )}}{{Θ}_{( k )}} [ \begin{matrix}{c}_{{δ}_{( k )}} ( {s}_{{θ}_{( k )}} - 1 ) \\ - {s}_{{δ}_{( k )}} ( {s}_{{θ}_{( k )}} - 1 ) \\ - {c}_{{θ}_{( k )}}\end{matrix} ] \\ and & ( 5 ) \\ {}_{}^{{b}_{( k )}}{R}{}_{g ( k )}^{} = {e}^{- {δ}_{( k )} [ \hat{w} x ]} {e}^{- {Θ}_{( k )} [ \hat{v} x ]} {e}^{{δ}_{( k )} [ \hat{w} x ]} & ( 6 )\end{matrix}$$

where \hat{v}=[0, 1, 0]^{T}, ŵ=[0, 0, 1]^{T} and the frames {g₍ₖ₎} and {b₍ₖ₎} are as shown in FIG. 7.

For

$${θ}_{( k )} = \frac{π}{2} ,$$

the formulation singularity,

$$\frac{1}{{Θ}_{( k )}} ,$$

resolves to

^{b}^{₍ₖ₎}P_{b}_{₍ₖ₎}_{g}_{₍ₖ₎}=[00L₍ₖ₎]^{T} (7) and

^{b}^{₍ₖ₎}R_{g}_{₍ₖ₎}=Iε [symbol] ^{3×3}. (8)

By differentiating Equations (5) and (6), the instantaneous direct kinematics takes the form

^{b}^{₍ₖ₎}t=J_{tψ}_{₍ₖ₎}\dot{ψ}₍ₖ₎

where, for

$${θ}_{( k )} \neq \frac{π}{2} ,$$

the Jacobian J_{qψ}_{₍ₖ₎} is given by

$${J}_{t {ψ}_{( k )}} = [ \begin{matrix}{Lc}_{{δ}_{( k )}} \frac{{Θ}_{( k )} {c}_{{θ}_{( k )}} - {δ}_{{θ}_{( k )}} + 1}{{Θ}_{( k )}^{2}} & - {Ls}_{{δ}_{( k )}} \frac{{s}_{{θ}_{( k )}} - 1}{{Θ}_{( k )}} \\ - {Ls}_{{δ}_{( k )}} \frac{{Θ}_{( k )} {c}_{{θ}_{( k )}} - {s}_{{θ}_{( k )}} + 1}{{Θ}_{( k )}^{2}} & - {Lc}_{{δ}_{( k )}} \frac{{s}_{{θ}_{( k )}} - 1}{{Θ}_{( k )}} \\ L \frac{Θ {s}_{{θ}_{( k )}} + {c}_{{θ}_{( k )}}}{{Θ}_{( k )}^{2}} & 0 \\ - {s}_{{δ}_{( k )}} & {c}_{{δ}_{( k )}} {c}_{{θ}_{( k )}} \\ - {c}_{{δ}_{( k )}} & - {s}_{{δ}_{( k )}} {c}_{{θ}_{( k )}} \\ 0 & - 1 + {s}_{{θ}_{( k )}}\end{matrix} ] \qquad ( 9 )$$

and, for

$${θ}_{( k )} = \frac{π}{2} ,$$

the formulation singularity, J_{qψ}_{₍ₖ₎}, is resolved by applying l'Hôpital's rule to yield

$${J}_{t {ψ}_{( k )}} = {[ \begin{matrix}- \frac{L}{2} {c}_{{δ}_{( k )}} & \frac{L}{2} {s}_{{δ}_{( k )}} & 0 & - {s}_{{δ}_{( k )}} & - {c}_{{δ}_{( k )}} & 0 \\ 0 & 0 & 0 & 0 & 0 & 0\end{matrix} ]}^{T} . \qquad ( 10 )$$

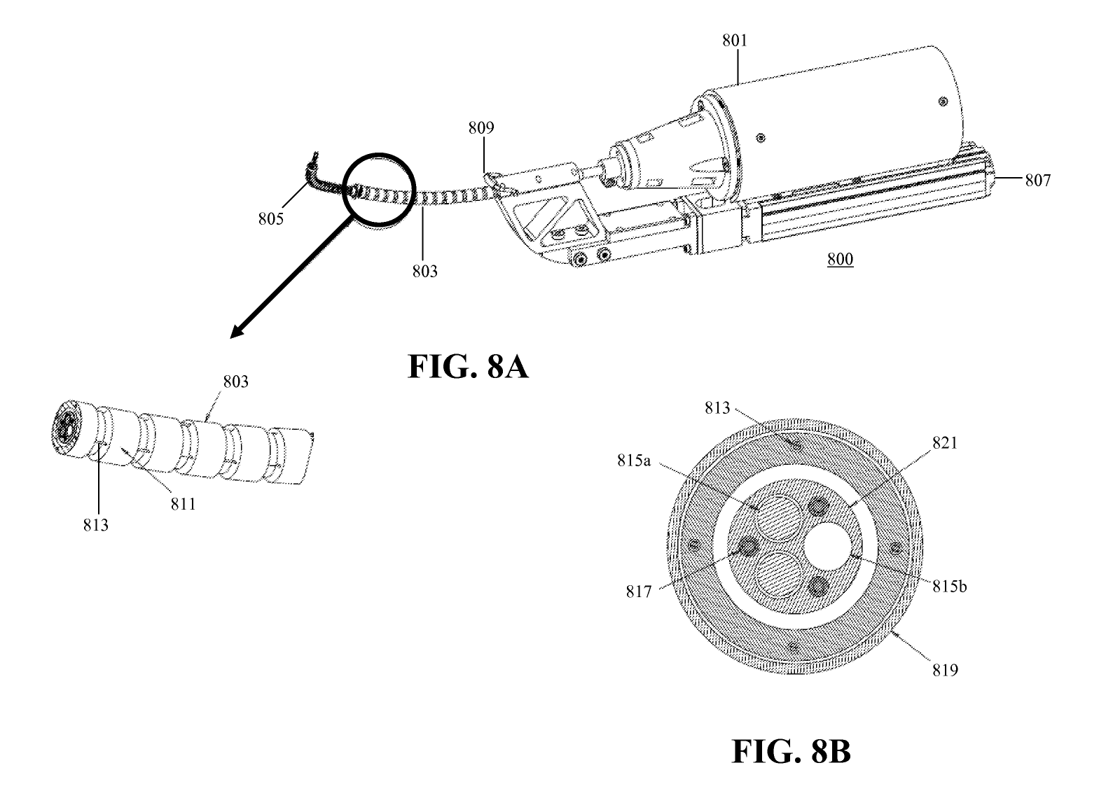

FIG. 8A is an illustration of a rapidly deployable flexible robotic system 800 for minimally invasive diagnosis and surgery in accordance with some embodiments of the disclosed subject matter. Referring to FIG. 8A, rapidly deployable flexible robotic system 800 includes an actuation unit 801, a concentric manual adjustment shaft 803, a continuum robot 805, a linear insertion stage 807, and a locking handle 809 for adjustment shaft 803.

In some embodiments, adjustment shaft 803 may allow rapid positioning of continuum robot 805 in a manner in which surgical and/or diagnostic instruments can be brought in and out of the surgical workflow rapidly. In some embodiments, adjustment shaft 803 may use a manually controlled internal cable tension such that the cable can be loosened when manual adjustment of adjustment shaft 803 is desired and tightened when an adjustment is complete.

In some embodiments, adjustment shaft 803 is constructed using a plurality of segments 811 and a set of locking cables 813. In some embodiments, locking cables 813 lock adjustment shaft 803 when tightened via a cam lock mechanism controlled by locking handle 809. In some embodiments, locking handle 809 may directly tighten locking cables 813 via a capstan.

FIG. 8B shows a cross section view of adjustment shaft 803 in accordance with some embodiments of the disclosed subject matter. Referring to FIG. 8B, adjustment shaft 803 includes locking cables 813, instrument channels 815a (carrying instrumentation), 815b (empty), secondary backbone lumens 817 for a continuum robot 805, a flexible plastic cover 819 (not shown in FIG. 8A) for adjustment shaft 803, and a flexible instrument housing 821. In some embodiments, flexible instrument housing 821 may be made of a PTFE extrusion.

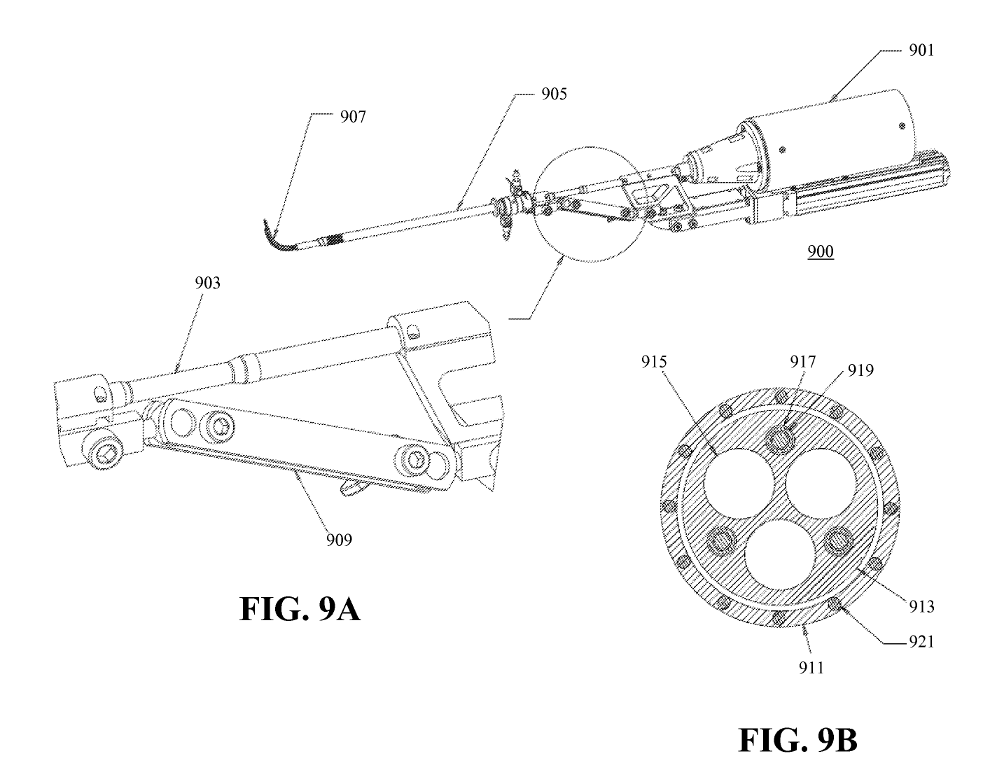

FIG. 9A is an illustration of a rapidly deployable flexible robotic system 900 for minimally invasive urologic procedures, such as transurethral resection of bladder tumors (TURBT), in accordance with some embodiments of the disclosed subject matter. Referring to FIG. 9A, rapidly deployable flexible robotic system 900 includes an actuation unit 901, a flexible shaft section 903, a urologic resectoscope 905, a continuum robot 907, and an adjustment arm 909.

In some embodiments, actuation unit 901 is equipped with force sensing capability. In some embodiments, flexible shaft section 903 is supported by adjustment arm 909 that is independent and separate from flexible shaft section 903. In some embodiments, adjustment arm 909 is manually adjustable and lockable. In some embodiments, adjustment arm 909 rigidly anchors at the proximal and distal ends of flexible shaft section 903 to support shaft section 903. In some embodiments, adjustment arm 909 is configured to couple to urologic resectoscope 905.

FIG. 9B shows a cross section view of flexible shaft section 903 in accordance with some embodiments of the disclosed subject matter. Referring to FIG. 9B, flexible shaft section is constructed of an internal lubricious structure including a PTFE extrusion 913 and an external supporting structure including a flexible support assembly 911. PTFE extrusion 913 in turn includes instrument channels/lumens 915 and secondary backbone lumens 917 for secondary continuum backbones 919 and flexible support assembly 911 includes support struts 921.

In some embodiments, support struts 921 may be made from stainless steel or other suitable flexible alloy. In some embodiments, adjustment arm 909 rigidly anchors only at the external support structure such that PTFE extrusion 913 can slide in the trajectory defined by the external supporting structure.

Together, adjustment arm 909 and flexible shaft section 903 provide for rapid deployment of robotic instruments into a surgical environment. Flexible shaft section 903 allows insertion of the robotic instruments without requiring the time consuming alignment of the inserted section of the robotic system 900 to a patient's urethra, thereby providing minimal disruption of the clinical work flow for deployment and removal of the robotic instruments.

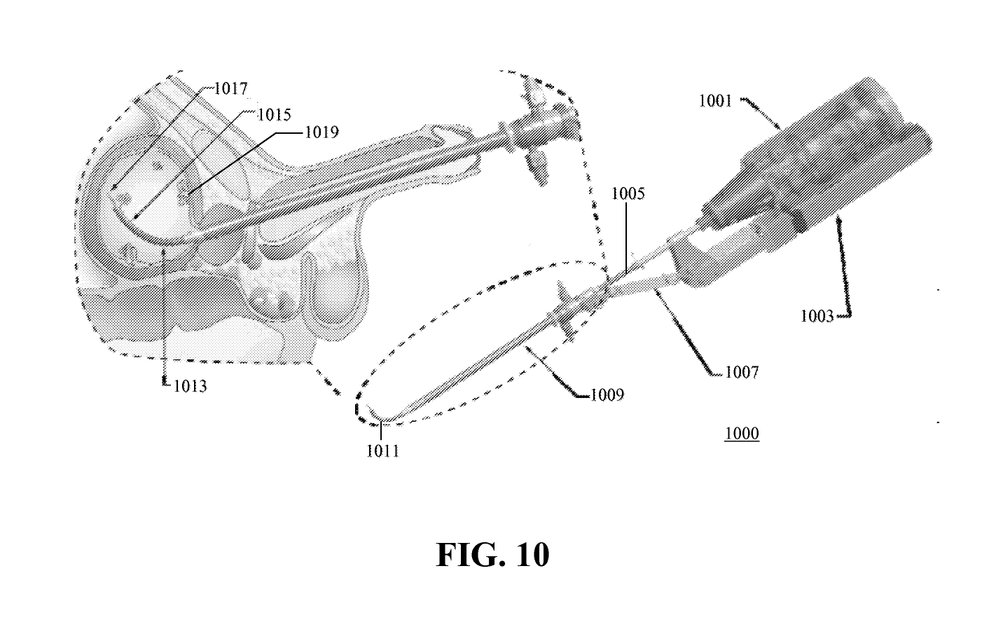

FIG. 10 is an illustration showing a deployment of a rapidly deployable flexible robotic system 1000 for performing transurethral resection of bladder tumors (TURBT) in a male patient in accordance with some embodiments of the disclosed subject matter. Referring to FIG. 10, robotic system 1000 includes an actuation unit 1001, a linear stage 1003, a flexible shaft section 1005, an adjustment arm 1007, a urethral resectoscope 1009, and a continuum robot 1011. The continuum robot 1011 includes a proximal continuum segment 1013 that is serially coupled to a distal continuum segment 1015 and carries, among other instruments, electrocautery loops 1017 and a fiberscope (not shown).

In order to perform a transurethral resection, in some embodiments, robotic system 1000 is fixed to a surgical bed and positioned relative to a patient with bladder cancer by advancing robotic system 1000 toward the patient's urethra using linear stage 1003. After robotic system 1000 is positioned near the urethra, the flexible shaft section 1005 is further adjusted using adjustment arm 1007 for rapid transurethral deployment.

Once resectoscope 1009 is transurethrally deployed, actuation unit 1001 is used to actuate continuum robot 1011 by manipulating proximal and distal continuum segments 1013 and 1015 to search for and reach the parts of bladder where suspicious tissue and visible lesions 1019 are located for resection using electrocautery loops 1017.

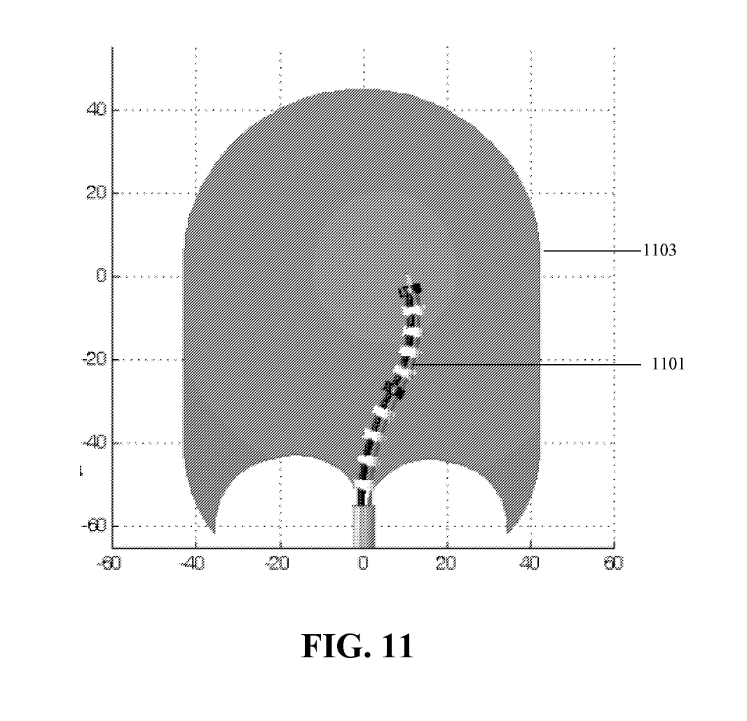

FIG. 11 is an illustration showing a cross section of a workspace 1103 that is reachable by a continuum robot 1101 of a rapidly deployable flexible robotic system, such as robotic system 1000, as illustrated in FIG. 10, in accordance with some embodiments of the disclosed subject matter.

Kinematics analysis was performed in a MATLAB computing environment to assess workspace 1103 relating to resection of tumors throughout the bladder. The analysis shows that a rapidly deployable flexible robotic system, such as robotic system 1000, is capable of visualizing and reaching throughout the bladder, including anterior aspects, as shown in FIG. 11.

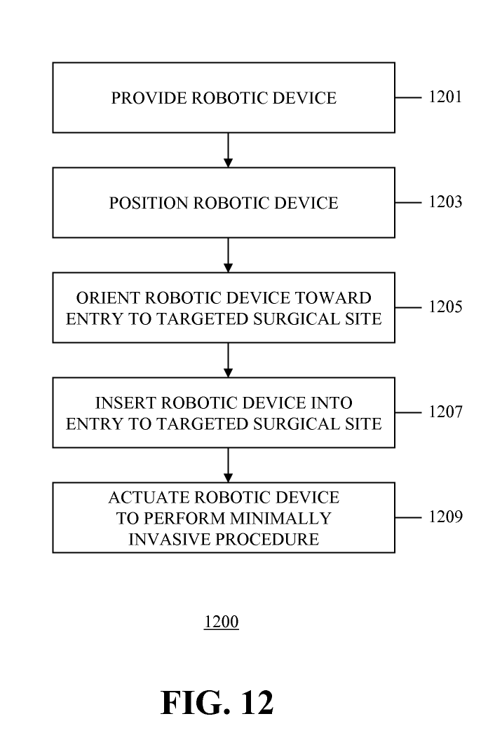

FIG. 12 is a flow chart for a method 1200 for deploying a robotic device for minimally invasive diagnosis and surgery in accordance with some embodiments of the disclosed subject matter. Referring to FIG. 12, a robotic device is provided at 1201. In some embodiments, the robotic device may be a rapidly deployable flexible robotic system, such as robotic system 300, 800 or 900, as illustrated in FIG. 3, 8, or 9, respectively. At 1203, the robotic device is positioned relative to a patient or a surgical bed. In some embodiments, the robotic device is fixed to a patient or a surgical bed via straps.

At 1205, the robotic device is oriented toward an entry to a targeted surgical site, such as urethra, oral opening, or an incision made near a targeted surgical site. In some embodiments, a flexible shaft that couples a continuum robot of the robotic device and an actuation unit for the robot is adjusted for rapid deployment of the robot. In some embodiments, an adjustment arm anchored at the proximal and the distal ends of the flexible shaft section is adjusted for orienting the robotic device to facilitate rapid deployment of the device into a restricted anatomy or opening. As shown in FIG. 10, for instance, adjustment arm 1007 may be used to manually adjust flexible shaft section 1005 to adjust the position and orientation of resectoscope 1009.

At 1207, the robotic device is inserted into an entry to a targeted surgical site. For a transurethral resection of bladder tumors, as shown in FIG. 10, for example, continuum robot 1011 coupled to urethral resectoscope 1009 is inserted into a patient's bladder through the patient's urethra.

At 1209, the robotic device is actuated to perform a minimally invasive procedure. For the transurethral resection of bladder tumors, for instance, suspicious tissue and visible lesions 1019 are resected using electrocautery loop 1017 and removed from the bladder.

Although the invention has been described and illustrated in the foregoing illustrative embodiments, it is understood that the present disclosure has been made only by way of example, and that numerous changes in the details of implementation of the invention can be made without departing from the spirit and scope of the invention. Features of the disclosed embodiments can be combined and rearranged in various ways.

1. A robotic system comprising:
a continuum robot for performing minimally invasive diagnostic, surgical or therapeutic techniques, wherein the continuum robot includes at least one continuum segment including a plurality of backbones, and wherein the continuum segment carries at least one diagnostic, surgical or therapeutic instrument in a flexible instrumentation housing that has a plurality of instrumentation channels;
an actuation unit for actuating the continuum robot by providing linear actuation to each of the plurality of backbones, wherein the actuation unit includes force sensors for measuring actuation forces; and
a flexible positioning shaft for directing a position and orientation of the continuum robot, wherein the flexible positioning shaft is configured to couple the actuation unit to the continuum robot.

2. The robotic system of claim 1, wherein the at least one instrument includes at least one of a surgical instrument and a visualization instrument.

3. The robotic system of claim 1, wherein the flexible positioning shaft is lockable and includes an internal structure and an external supporting structure that are coaxially arranged and wherein the flexible positioning shaft is separate and independent from the external supporting structure.

4. The robotic system of claim 3, wherein the internal structure of the flexible positioning shaft includes a lubricious poly-tetrafluoroethylene (PTFE) structure having a plurality of instrument channels and backbone lumens.

5. The robotic system of claim 3, wherein the external structures of the flexible positioning shaft includes a plurality of segments connected by a set of locking cables and wherein the locking cables lock the flexible positioning shaft when the locking cables are tightened.

6. The robotic system of claim 1, further comprising a linear insertion stage, wherein the actuation unit is mounted to the linear insertion stage.

7. The robotic system of claim 6, wherein at least a portion of the flexible positioning shaft is supported by the linear insertion stage.

8. The robotic system of claim 1, wherein the at least one continuum segment includes a proximal continuum segment that is serially coupled to a distal continuum segment.

9. A method for deploying a robotic device comprising:
providing a robotic device including an actuation unit, a flexible positioning shaft, and a continuum robot that is actuated by the actuation unit for performing minimally invasive procedures, wherein the robotic device is mounted on a linear stage and wherein the flexible positioning shaft is configured to couple the actuation unit to the continuum robot;
positioning the robotic device with respect to a surgical bed;
adjusting the flexible positioning shaft to orient the robotic device towards an entry to a targeted surgical site; and
inserting the robotic device into the entry by advancing the linear stage towards the entry.

10. The method of claim 9, wherein the continuum robot includes a proximal continuum segment that is serially coupled to a distal continuum segment and wherein the serially coupled segments carry at least one diagnostic, surgical or therapeutic instrument in a flexible instrumentation housing that has a plurality of instrumentation channels.

11. The method of claim 10, wherein the instrument channels includes at least one of a surgical instrument and a visualization instrument.

12. The method of claim 9, wherein positioning the robotic device includes fastening the robotic device to a surgical bed.

13. The method of claim 9, wherein the flexible positioning shaft is lockable and includes an internal structure and an external supporting structure that are coaxially arranged.

14. The method of claim 13, wherein the internal structure of the flexible positioning shaft includes a lubricious poly-tetrafluoroethylene (PTFE) structure having a plurality of instrument channels and backbone lumens and wherein the external structures of the flexible positioning shaft includes a plurality of segments connected by a set of locking cables and wherein the locking cables lock the flexible positioning shaft when the locking cables are tightened.

15. A robotic system comprising:
a continuum robot for performing minimally invasive urologic procedures, wherein the continuum robot includes a proximal continuum segment that is serially coupled to a distal continuum segment, and wherein the serially coupled segments include a plurality of backbones and carry at least one diagnostic, surgical or therapeutic instrument;
an actuation unit for actuating the continuum robot by providing linear actuation to each of the plurality of backbones, wherein the actuation unit includes force sensors for measuring actuation forces; and
a flexible shaft section for directing a position and orientation of the continuum robot, wherein the flexible shaft section is configured to couple the actuation unit to a transurethral resectoscope, wherein the transurethral resectoscope guides a flexible instrument housing and the at least one instrument from the actuation unit to the continuum robot, and wherein an adjustment arm rigidly anchors a proximal end and a distal end of the flexible shaft for adjusting the flexible shaft section to a desired position and orientation.

16. The robotic system of claim 15, wherein the at least one instrument includes at least one of a surgical instrument and a visualization instrument.

17. The robotic system of claim 16, wherein the surgical instrument includes biopsy forceps and the visualization instrument includes a fiberscope.

18. The robotic system of claim 15, wherein the urologic procedures include transurethral resection of bladder tumors.

19. The robotic system of claim 15, wherein the adjustment arm is lockable and the flexible shaft section includes an internal structure and an external supporting structure that are coaxially arranged.

20. The robotic system of claim 19, wherein the internal structure of the flexible shaft section includes a lubricious poly-tetrafluoroethylene (PTFE) structure having a plurality of instrument channels and backbone lumens.
